(19) United States
(12) Patent Application Publication
(10) Pub. No.: US 2026/0276634 A1
(43) Pub. Date: Sep. 17, 2026

(54) METHOD FOR DETERMINING INTESTINAL PERMEABILITY

(71) Applicant: BIOMEDAL, SL, Sevilla (ES)

(72) Inventor: Ángel CEBOLLA RAMÍREZ, Sevilla (ES)

(73) Assignee: BIOMEDAL, SL, Sevilla (ES)

(21) Appl. No.: 19/163,920

(22) PCT Filed: Mar. 15, 2024
(86) PCT No.: PCT/EP2024/057000

§ 371 Date: Sep. 10, 2025

(30) Foreign Application Priority Data
Mar. 16, 2023 (ES) P202330222

(51) Int. Cl. G01N 33/543; G01N 1/40; G01N 33/68

(52) U.S. Cl. CPC G01N 33/54388; G01N 1/4077; G01N 33/54306; G01N 33/6893; G01N 2001/4088; G01N 2333/415; G01N 2800/065; G01N 2800/52

(57) ABSTRACT

The present invention relates to an in vitro method for evaluating the state of intestinal permeability of a subject and, consequently, for the diagnosis of diseases or dysfunctions associated with intestinal hyper-permeability. More specifically, the procedure allows measuring using a common food component the amount of dietary antigen that can traverse a dysfunctional intestine. The procedure allows for the development of analytical products and processes within the framework of the medical devices industry.

### FIELD OF THE INVENTION

The present invention belongs to the medical field, clinical analysis, production and commercialization of health products and services, as well as basic and clinical research on the digestive system. Particularly, the present invention refers to an in vitro method for assessing the intestinal permeability status of a subject and, consequently, for the diagnosis of diseases or dysfunctions associated or that may occur with increased intestinal permeability such as (non-exhaustive list): celiac disease, Crohn's disease, irritable bowel syndrome, microscopic colitis, functional dyspepsia, chronic/acute villous atrophy due to chronic treatments with some drugs, and/or non-IgE-mediated food hypersensitivity.

### PRIOR ART

The intestinal barrier is fundamental to human health and constitutes the interface between the external environment and the internal milieu of the body. A functional intestinal barrier allows for the absorption of nutrients and liquids, while simultaneously blocking harmful substances such as toxins, bacteria, or viruses from crossing the intestinal epithelium and causing damage to the organism. Altered intestinal permeability has been associated with various chronic conditions such as inflammatory bowel disease, irritable bowel syndrome, celiac disease, and other pathologies originating in the gastrointestinal tract, as well as diseases like Alzheimer's and Parkinson's.

The pathways for the penetration of inadequately digested antigens or even bacteria and/or viruses are the paracellular route (through the intercellular space between cells) or the transcellular route (passing through the epithelial cells of the intestine). Increased intestinal permeability to macromolecules is associated with a variety of inflammatory conditions.

The luminal side of the intestine is lined with epithelial cells, which promote the absorption of water and nutrients; the epithelium also provides a dynamic and semipermeable barrier between the luminal microbiota and the host. The barrier is formed by membranes of individual epithelial cells and tight junction proteins that seal the paracellular space between adjacent cells. Therefore, the permeability of this barrier is regulated by the integrity of the cellular plasma membranes and tight junctions, as well as by the processes of epithelial cells mediating secretion and absorption. Small molecules (<300 Da) and electrolytes passively cross the tight junction barrier.

Membrane permeability can be altered by some physiological and pathological stimuli. During homeostasis, the intestinal epithelium absorbs nutrients while effectively preventing the translocation of luminal bacteria. However, some pathological conditions (e.g., intestinal toxins or inflammation) can increase paracellular transport and increase intestinal barrier permeability, presenting the risk of ineffective nutrient absorption and failure to prevent the translocation of luminal bacteria and their products. This can lead to chronic intestinal diseases, as well as affecting distal organs that drain and filter translocated bacteria and associated products.

Intestinal hyperpermeability may cause systemic inflammation, which is associated to various clinical conditions including:
Food allergies or sensitivities.
Gluten sensitivity and celiac disease.
Inflammatory bowel disease (Crohn's disease or ulcerative colitis).
Autoimmune diseases (rheumatoid arthritis, psoriasis, type I diabetes, spondylitis, etc.).
Cognitive dysfunction (anxiety, depression, schizophrenia, etc.).
Neurological conditions (Guillain-Barré syndrome, multiple sclerosis, etc.).

The known methods for determining intestinal permeability are:
1. The lactulose/mannitol or lactitol/mannitol test, which has been around for over 40 years and measures the ability of two sugar molecules or derivatives to cross the intestinal epithelium: lactulose (an isomer of lactose) and mannitol. Mannitol is a small sugar molecule that is easily absorbed, while lactulose is a larger molecule that is not effectively absorbed. After collecting a baseline urine sample, the patient ingests lactulose and mannitol. The concentration of lactulose and mannitol in the urine collected during the following 6 hours after ingestion is then quantified. It is an economical and easily performed method, as urine samples can be collected at the patient's home. However, it has the disadvantage of measuring the permeability of small sugar molecules that do not have immunogenic activity, furthermore, it does not allow for the analysis of the ability of antigenic macromolecules to pass through the epithelial barrier, which cause and exacerbate underlying inflammatory conditions and autoimmune diseases. Lactulose also has a low molecular weight, and the transfer of this substance through the intestinal barrier does not reflect the transfer of food proteins and the overall immune response. In addition, the ingestion of lactulose can exacerbate symptoms associated with small intestinal bacterial overgrowth (SIBO) and is also considered a prebiotic. Moreover, it requires techniques that required highly trained experts and/or expensive equipment, such as high-performance liquid chromatography (HPLC), gas chromatography, or tandem mass spectrometry (MS/MS). Finally, it is necessary for the patient to follow a complex diet due to the high presence of mannitol naturally occurring in plant-based foods (fruits, vegetables, legumes, among others).
2. Macromolecular indicators of intestinal permeability, which allow for the assessment of molecules that may be antigenic. The presence of specific antibodies against these molecules could reveal a possible increase in intestinal permeability in some past events. Among them are the followings:
   a. Lipopolysaccharides (LPS) are large molecules found in the outer membrane of gram-negative bacteria. They are endotoxins, and if absorbed, they provoke a strong immune response. Detection by LPS antibodies indicates the infiltration of endotoxins through the intestinal barrier into the systemic circulation.
   b. Occludin is a major component of the proteins that maintain tight junctions between intestinal cells. Detection by antibodies against occludins would indicate disruption of tight junctions (paracellular route).
   c. Zonulin has been proposed that it is a protein that regulates intestinal permeability. Detection by antibodies against zonulin indicates that tight junction regulation is impaired (paracellular route).
   d. The actomyosin network is a protein complex that regulates the function of the intestinal barrier by maintaining the plasticity of tight junctions. Antibodies against actomyosin are biomarkers of intestinal barrier dysregulation via cellular infiltration (transcellular route).
   e. LBP (lipopolysaccharide-binding protein). It has been determined that this protein binds to LPS and could be measured in blood.

It has been suggested that these indicators allow the identification of the level of permeable intestine and its causes (LPS or bacterial overgrowth vs. stress or food sensitivity).

Generally, the larger the molecule, the less likely it is to access the circulatory system. Antibody measurements are influenced by the use of steroids. Therefore, the patient has to stop using them orally or topically for at least 60 days before the analysis. Reference values are also only based on adults, making it difficult to apply when used for pediatric populations because there are no known normal value ranges.
3.—Direct measures of serum or blood zonulin. Circulating zonulin (vs. antibodies against zonulin) does not show false negatives with the use of oral or topical steroids, but its level could increase with the use of corticosteroids, leading to potential false positives for intestinal permeability. Zonulin levels are high in obese individuals and in glucose intolerance, showing potential false positives for intestinal permeability.

Therefore, to report the most important information, a convenient method to assess the state of intestinal permeability should be based on immunogenic proteins or peptides from commonly ingested foods to monitor diseases associated with permeability changes.

The present invention aims to solve these technical problems related to current methodologies which do not measure the capacity of entry of food antigenic peptides, are more invasive, expensive, indirect, and/or laborious, and proposes a new strategy for assessing the intestinal permeability status of a subject.

### DESCRIPTION OF THE INVENTION

### Brief Description of the Invention

The present invention refers to an in vitro method for assessing the state of intestinal permeability of a subject and, consequently, for the diagnosis of diseases or dysfunctions associated with or presenting with increased intestinal permeability including diseases such as celiac disease, Crohn's disease, irritable bowel syndrome, microscopic colitis, functional dyspepsia, chronic/acute villous atrophy due to chronic treatments with certain drugs, non-IgE-mediated food hypersensitivity, and also as risk factors for some neurodegenerative diseases such as Alzheimer's, Parkinson's, or multiple sclerosis. Currently, it is not possible to satisfactorily determine individual reference values and their alterations in intestinal permeabilization to large molecules such as proteins or large food peptides with immunogenic capacity. Thus, the present invention refers to an in vitro method for determining the degree of intestinal permeability, through the following general sequential procedure:
Step 1: Maintain a gluten-free diet for at least 16 hours, preferably 32, with at least the last 8 hours in fasting.
Step 2: Ingest a defined amount of gluten powder in suspension or hydrolyzed form (at least 0.5 g, preferably 10 g) in a fluid medium (water, shake, yogurts, etc.).
Step 3: Collect all urine for at least 6 hours, during which at least 4 hours continue to maintain fasting, and the gluten-free diet for all the period of urine collection.
Step 4: Measure the volume of collected urine and the amount of gluten in the collected urine using a procedure that detects less than 15 ng/ml of gliadin (as a protein used as an analytical standard for gluten) in urine.
Step 5: Compare the values of excreted gluten with internal reference values (of the same individual) or averages of healthy individuals and other reference values with altered gluten peptide permeability. If there are more gluten peptides in the urine than the internal reference value, it means that the individual has not improved their intestinal permeability. If compared with the corresponding average of their reference study group (age, gender) and it gives a higher value of gluten peptides than the normal margins, the individual will have increased intestinal permeability. If it is within the reference margins, the intestine shows no signs of increased permeability.

The previous gluten-free diet in step 1 is performed to ensure that all gluten peptides originate from the gluten ingested in suspension in step 2, not from food consumed in the hours before ingestion. The 8 hours of fasting prior to ingestion also ensure that the stomach is empty of food residues, as well as the part of the small intestine that connects to the jejunum, i.e., the duodenum. Prolonging fasting for more than 8 hours decreases the likelihood of interference with other foods in the metabolism of ingested gluten in step 2. Eliminating gluten from the diet for at least 24 hours before ingestion decreases the chances of having gluten residues from other previously ingested foods, although a restriction of 16 hours is usually sufficient to eliminate all detectable gluten in urine by commercially available methods (for example, the Gluten Detect by Biomedal, Seville).

The administered gluten is provided in amounts of at least 0.5 g to ensure its detection in urine and preferably purified (>80%) in powder or hydrolyzed form to increase the speed and efficiency of digestion and absorption by the intestine due to its high contact surface with the digestive system's hydrolytic agents. To facilitate ingestion in the analyzed individuals and make it more pleasant, the gluten suspension could be made in water using dispersing and flavoring agents, or in other fluids such as shakes, yogurt, etc. The gluten can be hydrolyzed in a pepsin-trypsin enzymatic hydrolysis or in an enzymatic digestion that allows solubilizing it without destroying most of the immunogenic gluten peptides (GIP) that can be detected by existing immunological methods on the market. Preferably, a gluten preparation of 4 to 40 g should be used to have gluten peptide levels in urine high enough to quantify GIP with commercially available methods, such as quantified GIP Urine lateral flow strips (LFIA) iVYCHECK read by a LFIA reader (Biomedal, Spain), and other methods that can be applied in small clinical laboratories and even points close to the patient (Point of Care).

After gluten ingestion, it is preferable for the patient to remain fasting for a period of 2 to 9 hours, favoring the rapid digestion of ingested gluten to produce the fragmented gluten peptides that will be translocated through the intestine.

Simultaneously with maintaining the gluten-free diet, all urines after the ingestion of the gluten preparation are collected for a fixed period of at least 8 hours, but extendable up to 16 hours in cases where it is necessary to collect most of the gluten peptides excreted in urine, so that those excreted subsequently are residual. Ideally, the collection period after ingestion would be urines from 6 to 8 in the morning until 22:00-00:00 at night, with a previous gluten-free diet for 24-36 hours and a minimum 8-hour fasting. The volume of excreted urine in that period is measured using a graduated urine collection container or a graduated cylinder. From that urine collected for 8 to 16 hours, the concentration of gluten peptides would be determined using a quantitative method.

In a preferred form of the invention, the methods for determining gluten peptides could use specific antibodies for gluten proteins already described, especially those that have already been shown to quantitatively determine gluten in food with high sensitivity (capable of detecting less than 15 ng/ml of gliadin) and have demonstrated their ability to detect gluten peptides in urine, such as G12, A1 (Biomedal, Spain), and R5 (r-Biopharma, Germany, or Eurofins Ingenasa, Spain). They could also be obtained by carrying out the usual procedures for obtaining antibodies, immunizing animals with peptides with those sequences that were most abundantly found when gluten peptides in urine were analyzed by liquid chromatography and mass spectrometry (LC/MS) (Table 1).

TABLE 1

| Name | Sequence |
|---|---|
| SEQ ID NO: 1 | LQLQPFPQPQLPYPQPQLPYPQPQLPYPQPQPF |
| SEQ ID NO: 2 | QPQLPY |
| SEQ ID NO: 3 | QPQLPF |
| SEQ ID NO: 4 | QPQLPL |
| SEQ ID NO: 5 | QQPFPQP |
| SEQ ID NO: 6 | QLPYPQP |
| SEQ ID NO: 7 | QLPFPQ |
| SEQ ID NO: 8 | QQPYPQP |
| SEQ ID NO: 9 | QPQPLY |
| SEQ ID NO: 10 | FPLQPQQP |
| SEQ ID NO: 11 | FPLEPEEP |

Antibodies obtained would be valid as long as the quantification limits of the analytical procedure are preferably below 15 ng/ml of gliadin, although very low concentrations of gluten peptides in urine require detection limits below 6 ng of peptide SEQ ID No. 1 (33-mer of α-gliadin)/ml to be able to detect the level of specific gluten peptides in urine after ingestion of 0.5-5 g of gluten.

To prepare the urine sample and increase detection sensitivity, peptide concentration systems in urine could also be applied in one form of the invention. Some preparative concentrations of gluten peptides that have proven effective include solid-phase extraction using matrices with C18, size exclusion chromatography, solvent evaporation, affinity chromatography, etc.

Preferred methods for detecting gluten in urine would include sandwich ELISA quantifying the amount of antigen-antibody complexes by coupling one of the specific antibodies to an enzyme that can use colorimetric, fluorimetric, or chemiluminescent reagents to send a measurable signal proportional to the formation of immunocomplexes with gluten peptides. Fluorimetric or chemiluminescent methods are indeed more convenient for application due to their wider dynamic range and do not usually require dilutions to enter ranges as when using colorimetric methods.

Also contemplated in a preferred embodiment of the invention would be lateral flow immunochromatographic strips that could be quantified with a strip reader by image analysis with gold or colored polystyrene particles, using classic or time-resolved fluorescence probes such as those achieved with europium particles, which could be quantified by an appropriate fluorimetric device. Other options like Heatsens or Quantum dots could be used as signal quantification options by coupling the appropriate probe to the antibody.

In an alternative embodiment of the invention, instead of antibodies, aptamers developed to recognize gluten in biological matrices could also be used, as has already been developed for food.

The invention procedure could be facilitated in a preferred form with a kit containing gluten powder in gelatin capsules, or in sachets with dispersing and/or flavoring agents containing at least 0.5 g of gluten, preferably between 4 and 40 g of gluten, and more preferably between 8 and 10 g of gluten. With such a quantity of gluten, gluten excretion is detectable by at least one of the commercially available methods. For example, by LFIA methods already marketed by Biomedal (iVYCHECK GIP Urine) based on sensitive and specific antibodies against the 33-mer of alpha-gliadin, achieving levels in urine after ingestion of 0.5 g above the detection limit in most of individuals (87%). It has been described that 0.5 g of gluten does not cause symptoms in most celiacs (Burger, J. P. W.; van Lochem, E. G.; Roovers, E. A.; Drenth, J. P. H.; Wahab, P. J. Dose-Escalating (50-500 mg) Gluten Administration Leads to Detectable Gluten-Immunogenic-Peptides in Urine of Patients with Coeliac Disease Which Is Unrelated to Symptoms, a Placebo Controlled Trial. Nutrients 2022, 14, 1771. https://doi.org/10.3390/nu14091771), therefore, these amounts could be used in cases of celiacs who fear the appearance of symptoms. In any case, for maximum efficacy of the invention procedure, it is recommended to use at least 4 g to yield a sufficient amount of gluten peptide to facilitate the detection in most individuals regardless of the degree of intestinal permeability due to the demand of sensitivity to detect the gluten peptides in urine. Population studies of celiac disease showed that they do not have a higher risk of mortality than the normal population. Gluten intake is frequent even in most celiacs, as between 67 and 89% of celiacs ingest gluten at least once over a week or a month, respectively [Silvester J A, Comino I, Rigaux L N, et al. Exposure sources, amounts and time course of gluten ingestion and excretion in patients with coeliac disease on a gluten-free diet. Aliment Pharmacol Ther. 2020; 00:1-11. https://doi.org/10.1111/apt.16075]. Occasional gluten intakes seem to be less harmful than a lower, but more frequent intake [Garzón-Benavides M, Ruiz-Carnicer Á, Segura V, Fombuena B, Garcia-Fernandez F, Sobrino Rodriguez S, et al. Clinical utility of urinary gluten immunogenic peptides in the follow-up of patients with coeliac disease. Aliment Pharmacol Ther. 2023; 00:1-11. https://doi.org/10.1111/apt. 17417].

Other optional components of the kit could be at least one urine collection container for at least the next 8 hours after gluten ingestion, preferably between for 9 and 16 hours, with a capacity of up to 2 L. In general, only 1.2 L of total capacity among all containers should be sufficient for that period of time (up to 12 hours since gluten intake). Gluten peptides are stable in urine and could be sent at room temperature to the analysis laboratory. Finally, another kit component would be the method of determining gluten peptides. The possibility of using LFIA for quantification would allow it to be performed as a Point-of-Care device (POC), in healthcare centers close to the patient with a reader that could be added in a preferred form of the invention, although quantitative laboratory methods such as ELISA, Luminex®, CLIA (chemiluminescence immunoassays), biosensors, etc., can be used.

In a more advanced form of the invention procedure, gluten peptides in urine can be concentrated by solid phase extraction, precipitation/centrifugation using specific formulations, or by size exclusion chromatography. For instance, gluten peptides can be fractionated by size exclusion chromatography (SEC) or by using molecular size filters such as Centricon®, Amicron®, or any device that can separate peptides corresponding to two molecular sizes. Preferably, the procedure would have between a minimum of 1 kDa (to be detectable by immunological methods and contain two epitopes) and a maximum of 30 kDa, the order of magnitude of the size of gliadin. The ratio between the total gluten peptide in urine and that in each fraction would be a factor that would allow knowing the amount of high molecular size peptides in urine, and consequently the degree of intestinal permeability to peptides of a certain size. These additional detailed studies could be carried out in individuals where more information is required to determine their enteropathy or degree of evolution of intestinal permeability.

The amount of gluten peptides in the urine of these individuals will be measured by an immunological method with specific antibodies that quantify gluten peptides resistant to intestinal digestion with epitopes capable of being detected in the urine of these individuals. The amount of gluten peptides excreted in urine could be compared with reference values. These reference values would be generated from tests with a population without digestive pathologies or conditions that impair their intestinal permeability. Alternatively, the values obtained in the first assay with an individual undergoing the invention procedure may be a personalize reference value to consider in determining the evolution of their intestinal permeability over a treatment or chronic disease being evaluated or monitored. A greater amount of gluten peptides detected compared to baseline or reference values would indicate increased intestinal permeability, while a decrease in these values would indicate normalization of intestinal permeability.

So, the first embodiment of the present invention refers to an in vitro method for assessing the state of intestinal permeability of a subject comprising: a) Administering orally to the subject at least 0.5 g of gluten (preferably between 0.5 and 40 g of gluten), or a source thereof, after a fasting period of at least 8 hours, and after a gluten-free diet of at least 16 hours, b) determining the total amount of gluten peptides present in a total urine sample obtained from the subject during at least the following 6 hours (preferably 6 to 16 hours) from the administration performed in step a), and c) wherein the determination in step b) of an amount of gluten significantly higher than a pre-established reference value, is indicative of intestinal hyper-permeability.

The second embodiment of the present invention refers to an in vitro method for the diagnosis, prognosis or monitoring patients suffering from diseases associated with intestinal hyper-permeability selected from the group consisting of: leaky gut syndrome, celiac disease, Crohn's disease, irritable bowel syndrome, microscopic colitis, functional dyspepsia, chronic/acute villous atrophy due to chronic treatments with certain drugs, systemic inflammation, autoimmune diseases and/or non-IgE-mediated food hypersensitivity, comprising determining intestinal permeability in a subject following the method of the first embodiment of the invention.

In a preferred embodiment of the invention, the gluten source has been purified and suspended in an aqueous fluid media.

In a preferred embodiment of the invention, the determination of the amount of gluten peptides is carried out directly through an immunoassay for the detection of the peptides themselves.

In a preferred embodiment of the invention, the immunoassay comprises antibodies against gluten peptides comprising the following sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and/or their derivatives.

In a preferred embodiment of the invention, the determination of the amount of gluten peptides is carried out through an immunoassay selected from: LFIA, ELISA, CLIA, SIMOA, Luminex, NULISA, turbidimetry or immunochromatography or biosensors.

In a preferred embodiment of the invention, the gluten peptides are concentrated by one of the following type of processes: solid phase extraction with hydrophobic resin, size exclusion chromatography, precipitation, or affinity purification.

The second embodiment of the present invention refers to a kit of parts suitable for carrying out the method of the invention comprising: a) Formulation with at least 0.5 g (preferably between 0.5 and 40 g) of purified gluten (preferably intact or partial hydrolyzed gluten powder for suspension or a ready to use solution or suspension), b) urine collection container and c) immunological test comprising antibodies capable of forming complexes with immunogenic gluten peptides and detectable by turbidimetric, visual, colorimetric, fluorimetric, chemiluminescent, and/or thermal detection methods.

In a preferred embodiment of the invention, the immunological test consists of a lateral flow strip with antibodies against immunogenic gluten peptides.

In a preferred embodiment of the invention, the immunological test consists of an ELISA, LFIA, CLIA or by magnetic or fluorescent or phosphorescent microparticles.

The third embodiment of the invention refers to gluten peptide characterized in that it comprises or consists of sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and/or their derivatives; or reactive antibodies against said peptides or their derivatives, for use in an in vitro method for evaluating the state of intestinal permeability of a subject. Alternatively, the present invention refers to the use of gluten peptide characterized in that it comprises or consists of sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and/or their derivatives; or reactive antibodies against said peptides or their derivatives, for evaluating in vitro the state of intestinal permeability of a subject.

In a preferred embodiment of the invention refers to gluten peptide or antibody for use in an in vitro method for assessing the intestinal permeability status of a subject comprising: (a) administering orally to the subject at least 0.5 of gluten (preferably between 0.5 and 40 g of gluten), or a source thereof, after a fasting period of at least 8 hours, and after a gluten-free diet of at least 16 hours, and (b) determining the amount of gluten peptides present in an urine sample obtained from the subject during at least the following 6 hours (preferably 6 to 16 hours) from the gluten ingestion, wherein the presence of an amount of gluten determined in step (b) significantly higher than pre-established reference values is indicative of intestinal hyper-permeability. Alternatively, the present invention refers to the use of gluten peptide characterized in that it comprises or consists of sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and/or their derivatives; or reactive antibodies against said peptides or their derivatives, for evaluating in vitro the state of intestinal permeability of a subject comprising: (a) administering orally to the subject at least 0.5 of gluten (preferably between 0.5 and 40 g of gluten), or a source thereof, after a fasting period of at least 8 hours, and after a gluten-free diet of at least 16 hours, and (b) determining the amount of gluten peptides present in an urine sample obtained from the subject during at least the following 6 hours (preferably 6 to 16 hours) from the gluten ingestion, wherein the presence of an amount of gluten determined in step (b) significantly higher than pre-established reference values is indicative of intestinal hyper-permeability.

In a preferred embodiment the invention of the invention comprises a concentration step by size exclusion chromatography in which an increase of the average size of urine gluten peptides is indicative of intestinal hyperpermeability of the gut.

In the context of the present invention, the following terms or expressions are defined for a better interpretation of its scope:
The term “comprising” means including, but not limited to, what follows the word “comprising”. Thus, the use of the term “comprising” indicates that the listed elements are necessary or mandatory, but that other elements are optional and may or may not be present.
The term “consisting of” means including, and limited to, what follows the phrase “consisting of”. Therefore, the phrase “consisting of” indicates that the listed elements are necessary or mandatory, and that no other elements can be present.
The term “pre-established reference values,” when referring to the quantity of gluten determined in the present invention, refers to the quantity observed in healthy individuals (i.e., subjects who are not suffering from intestinal hyper-permeability), particularly in the initial state of the same individual before treatment. In a preferred aspect, the reference value is measured in an initial assay performed on the subject, on the results of a population without enteropathies, or on the results of a population without enteropathies of the same age range as the individual whose intestinal permeability is to be analyzed. It is likely that the subject has increased intestinal permeability, with a certain sensitivity and specificity, if the quantity of gluten determined in the subject's urine is above said “pre-established reference level.” A “reference” value can be a threshold value or a cut-off value that can be determined experimentally, empirically, or theoretically. A threshold value can also be arbitrarily selected based on existing experimental and/or clinical conditions, as recognized by one skilled in the art. The threshold value must be determined to obtain optimal sensitivity and specificity according to the function of the test and the benefit/risk balance (clinical consequences of false positives and negatives). Typically, optimal sensitivity and specificity (and therefore the threshold value) can be determined by an ROC (Receiver Operating Characteristic) curve based on experimental data.

### BRIEF DESCRIPTION OF THE FIGURES

FIG. 1. Classification of the state of villous atrophy of the intestinal mucosa. VH:CD (Villus height, crypt depth) is the mean ratio between the height of the intestinal villus and the depth of the crypt. A ratio of 2 or higher is considered a state of the intestinal villi according to Marsh classification as 0 to 2, indicating no or mild intestinal atrophy. Ratios below 2 are considered Marsh 3, with degrees ranging from 3a to 3c, implying severe intestinal atrophy.

FIG. 2. Dependence of the concentration of immunogenic gluten peptides in urine and degrees of villous atrophy. All patients ingested 3 g of gluten contained in an energy bar. VH:CD is the ratio between villus height and crypt depth. LOQ=Limit of quantification (2.5 ng/ml).

FIG. 3. Comparison of the average excretion of GIP in celiac individuals according to the degree of villous atrophy in biopsies performed after the period of daily ingestion of 3 g of gluten extended for at least 2 months.

FIG. 4. Dynamics of gluten peptide excretion in individuals without known enteropathies after ingestion of 8 g of powdered purified gluten in fasting conditions. Each color represents a different volunteer.

FIG. 5. Dynamics of excretion of lactulose excreted from the total ingested (10 g) in individuals without known enteropathies (n=15), expressed in ppm. Each color represents a different volunteer.

FIG. 6. Correlation between GIP excretion and lactulose/mannitol excretion ratios in 15 hours total urine collection from the 15 volunteers after ingestion of 8 g of purified gluten suspension in previous fasting conditions. R²=0.51.

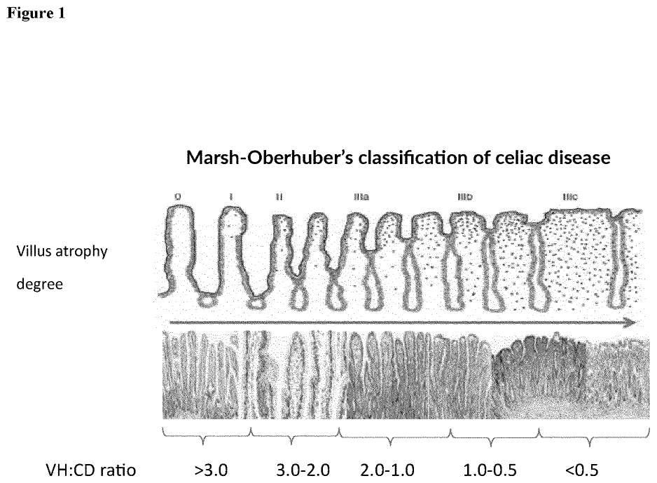

### DETAILED DESCRIPTION OF THE INVENTION

The present invention is illustrated by means of the Examples set below, without the intention of limiting its scope of protection.

### Example 1. Variation in Gluten Peptide Concentration in Urine in Celiac Individuals with Different Degrees of Villous Atrophy

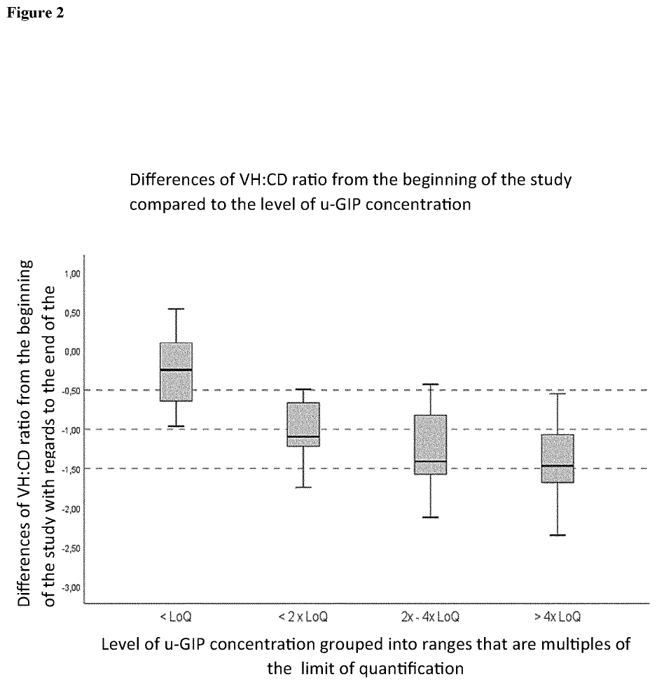

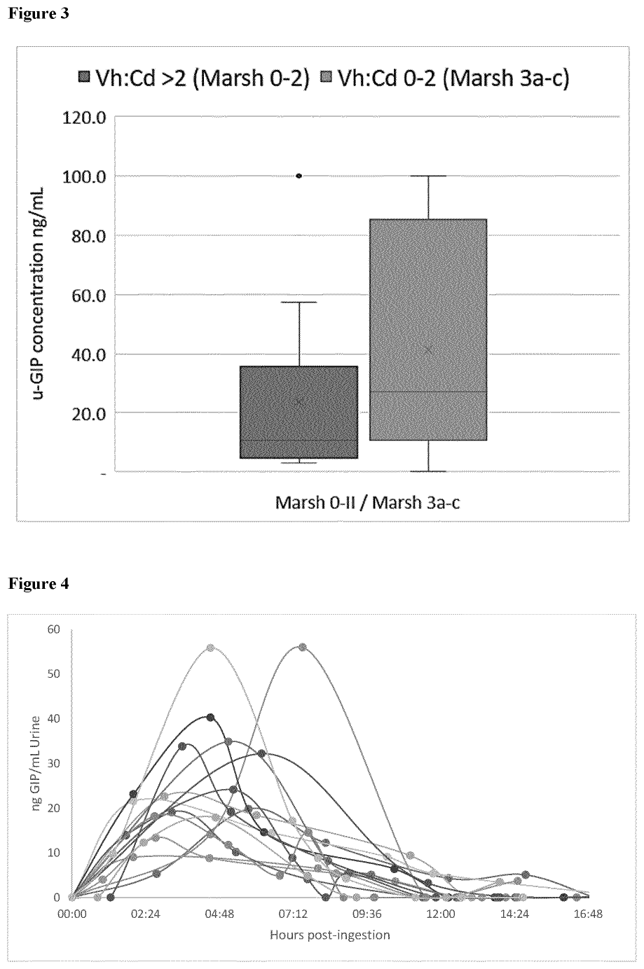

This example demonstrates how the results of clinical studies show that the degree of villous atrophy in celiac patients increases the values of GIP excretion in urine (u-GIP) for continuous intake, even without controlling the intake of other foods or the time of urine collection. Statistically significant variations in peptide concentration can be observed depending on the degree of duodenal villous atrophy. In this study, identical amounts of gluten were administered for a few days before or after a duodenal biopsy, which provided data on the degree of villous atrophy in celiac individuals by measuring the mean ratio of villous height to crypt depth (Vh:Cd). The analyzed subjects (celiac patients) received identical amounts of gluten (3 g in the form of an energy bar) daily for six weeks following a duodenal biopsy, which provided data on the degree of villous atrophy by measuring the mean ratio of villous height to crypt depth. It was observed that the concentration of detectable gluten immunogenic peptides in immunoassays was significantly higher the more atrophied the intestinal mucosa was. Therefore, for identical amounts of ingested gluten, the most determining factor in the concentration of gluten peptides excreted in urine was the degree of intestinal permeability increased by the increasing deterioration of the intestinal epithelium due to gluten intake (FIG. 2). The average gluten concentration in urine of celiacs with degrees of atrophy 0 to 2 was 23.7 ng/ml and their median 11.8 ng/ml. For those with Marsh 3, the average was 45.7 ng/ml and the median 29.9 ng/ml. Although the average deviation was high in both cases (21 and 33, respectively), the trend was significant suggesting more permeability in those with Marsh 3. Within those with Marsh 3, the mean and median of those with Marsh 3a, Marsh 3b, and Marsh 3c were seen. These results are shown in Table 2 and FIG. 3.

TABLE 2
GIP concentration in samples of patients with different degrees of
intestinal atrophy after ingestion of 3 g of gluten.VH:CD is the
villus height/crypt depth ratio.
Table 2

| | VH:CD | | |
|---|---|---|---|
| | 0-0.5 | 0.5-1 | <1 |
| Mean u-GIP ng/ml | 55.9 | 31.4 | 8.62 |
| Median u-GIP ng/ml | 49.8 | 23.9 | 9.2 |

Higher degrees of atrophy are associated with higher concentrations of GIP in urine, with the differences being particularly significant in cases of severe atrophy (Marsh 3b and 3c). The most feasible explanation was that the paracellular gut permeability was increased in gut mucosal damage due to gluten daily gluten intake.

### Example 2. Variability of Metabolism Between Individuals

This example of use of the invention shows how, in two different individuals, the values of metabolized and excreted gluten from different days give similar u-GIP values. For this purpose, individuals 1 and 2 were put on a gluten-free diet for 36 hours and at least 9 hours of fasting before taking a sachet of gluten (8 g) which was suspended in 250 ml of water. Before gluten intake, a GIP detection test in urine was performed to confirm negative results. After intake, they remained on a gluten-free diet for at least 8 hours, urine was collected for 9 hours, and subsequently the GIP concentration in urine was measured with a lateral flow test and the iVYCHECK Reader for quantification, calculating the volume of the same. In both analyzed individuals, a standard deviation of GIP excretion of less than 20% between measurements made on different days was detected (Table 3), therefore with the values of each individual may serve as an internal reference value prior to a treatment that improves the intestinal permeability of a given individual because variations in the absence of pathologies are not significant.

TABLE 3
Quantification of gluten peptide excretion after fasting intake of
8 g of gluten suspension on different days in two volunteers (μg
of gluten excreted in urine during the 9 hours following intake).
Table 3

| | | | | | Standard |
|---|---|---|---|---|---|
| | Day 1 | Day 5 | Day 10 | Mean | deviation |
| Volunteer 1 | 16.3 | 12.0 | 14.9 | 14.4 | 2.2 |
| Volunteer 2 | 26.0 | 21.9 | 21.3 | 23.1 | 2.5 |

The detected ratios of GIP in urine to those ingested are 14-23 μg detected per every 8 g of gluten ingested. About 8 g of gluten ingested using specific anti-33-mer immunoassay methods, are equivalent to around 1-1.33 g of GIP using iVYCHECK urine (Biomedal, Spain). The amount excreted therefore represents only 0.001-0.002% of the amount ingested, which is why a specific and highly sensitive technique is needed to detect these peptides in urine.

### Example 3. The Amount of Gluten Peptides Excreted in Different Individuals Compared to the Permeability of Lactulose/Manitol

In this example, it can be observed how the excretion of almost all detectable gluten in urine can occur within a few hours after fasting intake of a fixed amount of powdered gluten, and how it correlates better with the excretion of lactulose than with a smaller molecule like mannitol, indicating that we are estimating paracellular permeability (lactulose) versus intracellular permeability (mannitol). Additionally, it can be observed how the excretion of most of detectable gluten (>80%) occurs in the first 9 to 15 hours, reducing the degree of intraindividual variability with respect to lactulose. Specifically, subjects (n=15) received 8 g of gluten after at least 8 hours of fasting, following a gluten-free diet for at least 36 hours in individuals without diagnosed celiac disease. The gluten consisted of gluten powder with flavorings (sweeteners and food colorings) contained in single-use heat-sealed sachets. At the same time, volunteers took 1 g of mannitol and 10 g of lactulose. Subsequently, the various urines were collected in separate containers, to be able to perform individualized measurements of the concentration in each void, as well as the total volume of the urines. The amount of gluten peptides present in urine excreted for 15 hours following gluten intake was determined using the iVYCHECK urine product and a calibrated lateral flow strip reader iVYCHECK Reader (Biomedal, Spain). The ingested gluten was mixed with 1 g of mannitol and 10 g of lactulose for use as control. Mannitol allows estimation of intracellular permeability, and lactulose paracellular permeability, studying the reference values which is already widely described in the biomedical literature. The amount of gluten peptides correlated directly with intestinal permeability. Although factors such as the speed of gluten digestion can influence, it is understood that within the same individual the reference values can be constant, as seen in the previous example. Given the variability of gluten metabolism and its excretion kinetics, intake was recommended once the subject had urinated to empty the bladder as much as possible (early in the morning), preferably maintaining a fasting period of at least 8 hours for the accelerated metabolism of ingested gluten until its excretion without being affected by other simultaneously ingested foods. The obtained values could be compared with reference values obtained from conducting the same procedure with individuals with or without enteropathies associated with intestinal permeability, or they could be used as references themselves for subsequent determinations indicating evolution towards a higher value of gluten peptide excretion (higher permeability) or lower gluten peptide excretion (lower permeability).

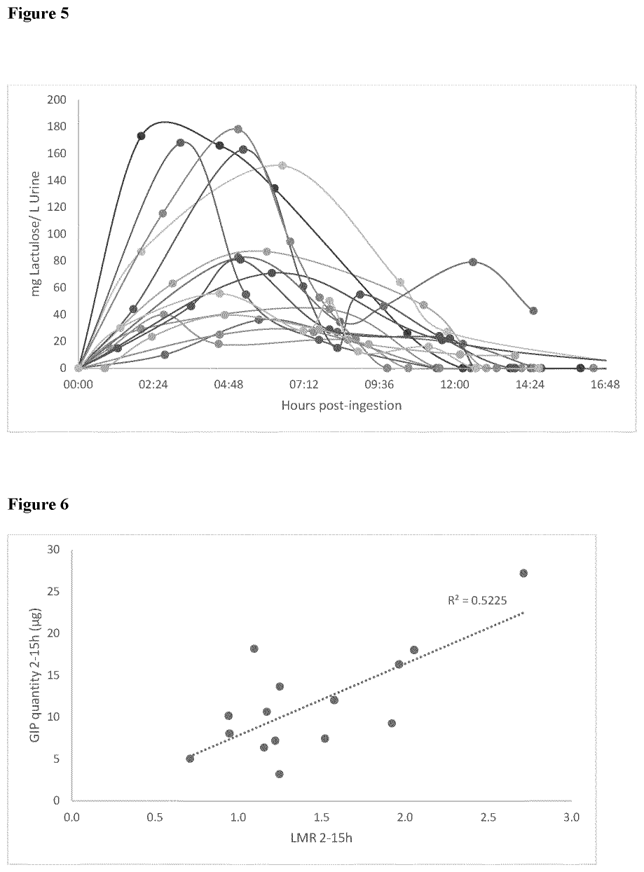

In this example conducted with 15 volunteers without symptoms of diagnosed digestive disease, basal urine is analyzed. All urines were collected in separate containers for 15 hours after the intake of the gluten sachet. The excreted urines were mixed: between 0-6 h after intake. Then between 2-15 h after intake. The concentrations of GIP in the excreted urines were measured, both by ELISA and LFIA between 0-6 h after intake and between 2-15 h after intake individually. The GIP concentration of each urine of each volunteer (FIG. 4) and lactulose (FIG. 5) were measured, and subsequently, the % excreted relative to the ingested GIP and the lactulose/mannitol ratio (FIG. 6) were calculated. The correlation between lactulose: mannitol and GIP was determined. The dynamics of lactulose excretion are different in each individual, with excretion occurring beyond 15 h. It is known that sugars can be reabsorbed by the body. That is why lactulose can have several peaks of excretion. It is estimated that lactulose and GIP both pass through the same route, the paracellular one. Therefore, the mixtures of 2-15 h of GIP are more similar to those of 0-6 h and 2-15 h of lactulose.

Analyses are also conducted to detect outliers in each group and eliminate them from the correlations.

1. In vitro method for assessing the state of intestinal permeability of a subject comprising:
a. Administering orally to the subject at least 0.5 g of gluten, or a source thereof, after a fasting period of at least 8 hours, and after a gluten-free diet of at least 16 hours,
b. Determining the total amount of gluten peptides present in a total urine sample obtained from the subject during at least the following 6 hours from the administration performed in step a),
wherein the determination in step b) of an amount of gluten significantly higher than a pre-established reference value, is indicative of intestinal hyper-permeability.

2. In vitro method for the diagnosis, prognosis or monitoring patients suffering from diseases associated with intestinal hyper-permeability selected from the group consisting of: leaky gut syndrome, celiac disease, Crohn's disease, irritable bowel syndrome, microscopic colitis, functional dyspepsia, chronic/acute villous atrophy due to chronic treatments with certain drugs, systemic inflammation, autoimmune diseases and/or non-IgE-mediated food hypersensitivity, comprising determining intestinal permeability in a subject following the method of claim 1.

3. In vitro method, according to any of the preceding claims, characterized in that the gluten source has been purified and suspended in an aqueous fluid media.

4. In vitro method, according to any of the preceding claims, characterized in that the determination of the amount of gluten peptides is carried out directly through an immunoassay for the detection of the peptides themselves.

5. In vitro method, according to any of the preceding claims, wherein the immunoassay comprises antibodies against gluten peptides comprising the following sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and/or their derivatives.

6. In vitro method, according to any of the preceding claims, characterized in that the determination of the amount of gluten peptides is carried out through an immunoassay selected from: LFIA, ELISA, CLIA, SIMOA, Luminex, NULISA, turbidimetry or immunochromatography or biosensors.

7. In vitro method according to any of the preceding claims, characterized in that the gluten peptides are concentrated by one of the following type of processes: solid phase extraction with hydrophobic resin, size exclusion chromatography, precipitation, or affinity purification.

8. In vitro method, according to any of the preceding claims, wherein a concentration step by size exclusion chromatography is carried out in which an increase of the average size of urine gluten peptides is indicative of intestinal hyperpermeability of the gut.

9. Kit of parts suitable for carrying out the method of claims 1 to 8 comprising:
a. Formulation with at least 0.5 g of purified gluten.
b. Urine collection container.
c. Immunological test comprising antibodies capable of forming complexes with immunogenic gluten peptides and detectable by turbidimetric, visual, colorimetric, fluorimetric, chemiluminescent, and/or thermal detection methods.

10. Kit of parts, according to claim 9, wherein the immunological test consists of a lateral flow strip with antibodies against immunogenic gluten peptides.

11. Kit of parts, according to any of the claim 9 or 10, wherein the immunological test consists of an ELISA, LFIA, CLIA or by magnetic or fluorescent or phosphorescent microparticles.

12. Use of a gluten peptide characterized in that it comprises or consists of sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and/or their derivatives; or reactive antibodies against said peptides or their derivatives, for evaluating in vitro the state of intestinal permeability of a subject.

13. Use of a gluten peptide or antibody for use, according to claim 12, for evaluating in vitro the state of intestinal permeability of a subject by: (a) administering orally to the subject at least 0.5 of gluten, or a source thereof, after a fasting period of at least 8 hours, and after a gluten-free diet of at least 16 hours, and (b) determining the amount of gluten peptides present in an urine sample obtained from the subject during at least the following 6 hours from the gluten ingestion, wherein the presence of an amount of gluten determined in step (b) significantly higher than pre-established reference values is indicative of intestinal hyper-permeability.

14. Use, according to claim 12 or 13, wherein a concentration step by size exclusion chromatography is carried out in which an increase of the average size of urine gluten peptides is indicative of intestinal hyperpermeability of the gut.


SEQUENCE LISTING (11 sequences)

SEQ ID NO: 1 - protein, 33 aa, synthetic construct
LQLQPFPQPQLPYPQPQLPYPQPQLPYPQPQPF

SEQ ID NO: 2 - protein, 6 aa, synthetic construct
QPQLPY

SEQ ID NO: 3 - protein, 6 aa, synthetic construct
QPQLPF

SEQ ID NO: 4 - protein, 6 aa, synthetic construct
QPQLPL

SEQ ID NO: 5 - protein, 7 aa, synthetic construct
QQPFPQP

SEQ ID NO: 6 - protein, 7 aa, synthetic construct
QLPYPQP

SEQ ID NO: 7 - protein, 6 aa, synthetic construct
QLPFPQ

SEQ ID NO: 8 - protein, 7 aa, synthetic construct
QQPYPQP

SEQ ID NO: 9 - protein, 6 aa, synthetic construct
QPQPLY

SEQ ID NO: 10 - protein, 8 aa, synthetic construct
FPLQPQQP

SEQ ID NO: 11 - protein, 8 aa, synthetic construct
FPLEPEEP